(19) United States
(12) Patent Application Publication
(10) Pub. No.: US 2011/0059085 A1
(43) Pub. Date: Mar. 10, 2011

(54) Staphylococcus aureus-specific antibody preparations

(75) Inventor: Stanley A. Kim, Wellington, FL (US)

(73) Assignee: STROX BIOPHARMACEUTICALS, LLC, Wellington, FL (US)

(21) Appl. No.: 12/944,380

(22) Filed: Nov. 11, 2010

Related U.S. Application Data
(63) Continuation-in-part of application No. PCT/US09/43545, filed on May 12, 2009.
(60) Provisional application No. 61/052,281, filed on May 12, 2008.

(51) Int. Cl. A61K 39/40; A61P 37/04; A61P 31/04

(52) U.S. Cl. 424/133.1; 424/165.1

(57) ABSTRACT

A new antibody-based strategy for treating or preventing Staphylococcus aureus infections utilizes IgM antibodies specific for one or more Staphylococcus aureus capsular antigens. Examples of such antibodies include (i) a polyclonal IgM antibody composition that is isolated from pooled donor plasma and enriched for those IgMs that specifically bind Staphylococcus aureus capsular antigens or (ii) one or more IgM monoclonal antibodies that specifically bind Staphylococcus aureus capsular antigens.

### CROSS-REFERENCE

The present application is a continuation-in-part under 35 U.S.C. §120 of co-pending International Application No. PCT/US09/043545, filed on May 12, 2009, which designated the United States and claims the priority of U.S. provisional patent application No. 61/052,281 filed on May 12, 2008.

### FIELD OF THE INVENTION

The invention relates generally to the field of antibodies (Abs), immunology, infectious diseases, and medicine. More specifically, the invention relates to Ab preparations enriched for immunoglobulins (Igs) of the IgM type specific for Staphylococcus aureus (SA) capsular antigens.

### BACKGROUND

SA is a substantial cause of sickness and death in both humans and animals. Infection with these gram-positive cocci often results in the development of a superficial abscess. Other cases of SA infection can be much more serious. For example, intrusion of SA into the lymphatics and blood can lead to a systemic infection which in turn can cause complications such as endocarditis, arthritis, osteomyelitis, pneumonia, septic shock and even death. Hospital-acquired SA infection is common and particularly problematic with SA being the most frequent cause of hospital-acquired surgical site infections and pneumonia, and the second most frequent cause of cardiovascular and bloodstream infections.

The economic impact of SA infection is considerable. A retrospective analysis by Noskin et al. (Arch Intern Med 165: 1756-1761, 2005) found that SA infection was reported as a discharge diagnosis for 0.8% of all U.S. hospital inpatients, and that, compared to inpatients without SA infection, those with SA infection had 3 times the length of hospital stay, 3 times the total charges ($48,824 vs. $14,141), and 5 times the risk of in-hospital death. Extrapolating these figures, Noskin et al. estimate that SA infection of U.S. hospital patients results in about 12,000 inpatient deaths and $9.5 billion in excess charges per year.

Antibiotic administration has been and remains the standard treatment for SA infections. Unfortunately, the use of antibiotics has also fueled the development of antibiotic resistance in SA. Notably, methicillin-resistant SA (MRSA) has evolved the ability to resist beta-lactam antibiotics such as penicillin and cephalosporins. More alarmingly, SA resistant to antibiotics of last resort such as vancomycin and linezolid have recently emerged. Therefore a new approach for preventing and treating SA infections is needed.

### SUMMARY

The invention relates to the development of a new Ab-based strategy for treating or preventing SA infections. This strategy utilizes IgM Abs specific for one or more SA capsular antigens. Examples of such Abs include (i) a polyclonal IgM Ab composition that is isolated from pooled donor plasma and enriched for those IgMs that specifically bind SA capsular antigens or (ii) one or more IgM monoclonal Abs (mAbs) that specifically bind SA capsular antigens.

Previous Ab-based strategies for treating or preventing SA infections showed promise in pre-clinical and early stage clinical trials, but failed to meet endpoints in phase III trials. Perhaps explaining these results, no previous strategy addressed both of the two major SA virulence factors which act synergistically to evade an Ab response, namely: (i) polysaccharide capsule production and (ii) staphylococcal protein A (Spa) expression. By coating the outer surface of the bacterial cell wall, a polysaccharide capsule “cloaks” other cell wall-associated SA antigens from being recognized by the Abs and immune effector cells which would otherwise attack the bacteria. Ab preparations specific for non-capsular SA antigens are therefore not very effective at killing the bacteria because the Igs in the Ab preparations are required to first penetrate the capsule in order to reach the non-capsular antigen. And even once the capsule is penetrated, the Igs bound to the non-capsular antigen are located under the capsule and out of reach of other immune effectors such as phagocytes. Aggravating this, Spa, a cell wall-associated protein that binds Igs via their Fc (effector) regions, acts as both (i) a sponge that absorbs those Igs that bind a capsular antigen or happen to non-specifically penetrate the capsule and (ii) an Fc region anchor that orients the effector portion of an Ig away from Fc-interacting immune effectors such as complement and Fc receptor-bearing phagocytes. Accordingly, even most Abs specific for SA capsular antigens are “sequestered” from immune effectors in this manner. Underscoring the importance of the synergistic effect of cloaking and sequestration, nearly all clinical isolates of SA express both of these virulence factors.

The Abs of the invention overcome this synergistic cloaking and sequestration defense by targeting an uncloaked antigen (i.e., the polysaccharide capsule itself) with Igs (e.g., IgMs) that are not (or are only poorly) sequesterable by Spa. IgM Abs are currently preferred because they have other characteristics (e.g., efficient complement activation) that make them very effective at clearing encapsulated bacteria. Indeed, a deficiency in IgM memory B cells such as occurs in asplenia is correlated with an impaired immune response to encapsulated bacteria.

Accordingly, in one aspect, the invention features a composition including a polyclonal mixture of Abs purified from blood plasma pooled from at least ten different donors (e.g., at least 5, 10, 20, 100, 500, 1000 of more L of pooled plasma), wherein the mixture of Abs is enriched for non-Spa-binding Igs that specifically bind at least one SA capsular antigen. The mixture of Abs can be enriched for IgMs (e.g., at least about 1, 2, 3, 4, 5, 10, 30 or more percent by weight of the mixture of Abs) that specifically bind the at least one SA capsular antigen. The at least one SA capsular antigen can be a serotype 5 capsular antigen, a serotype 8 capsular antigen, or a mixture of both of the foregoing. In certain embodiments, the Abs of the composition are human. In other embodiments, the Abs are bovine (for treating SA-associated bovine mastitis). The plasma donors can be those have or have not been immunized with a vaccine including an agent that induces an immune response against SA (e.g., at least one SA capsular antigen).

The mixture of Abs can lyophilized; dissolved in a solution including sodium and chloride ions; dissolved in a solution including one or more stabilizing agents such as albumin, glucose, maltose, sucrose, sorbitol, polyethylene glycol, and glycine; and/or dissolved in a solution including a microbicide (e.g., a detergent, an organic solvent, and a mixture of a detergent and organic solvent).

In another aspect, the mixture of Abs can also be enriched for Igs that specifically bind one or more (e.g., 1, 2, 3, 4, 5, 6, 7, 8, 9, 10 or more) SA antigens other than a capsular antigen. Examples of such other antigens include Spa, the serotype 336 polysaccharide antigen, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, and Sbi.

Also within the invention is a method including the step of isolating from pooled plasma a polyclonal mixture of Abs that is enriched for non-staphylococcal protein A-binding Igs that specifically bind at least one SA capsular antigen. This method can further include the step of dissolving the isolated polyclonal mixture of Abs in a pharmaceutically acceptable carrier, filtering the isolated polyclonal mixture of Abs, contacting the isolated polyclonal mixture of Abs with a detergent, contacting the isolated polyclonal mixture of Abs with an organic solvent, contacting the isolated polyclonal mixture of Abs with β-propiolactone, and/or subjecting the isolated polyclonal mixture of Abs to dialysis.

Another method within the invention includes the steps of: (a) obtaining plasma pooled from at least ten donors; (b) separating the pooled plasma into at least a first portion and a second portion, the second portion being enriched for non-Spa-binding Igs compared to the first portion; (c) separating the second portion into at least a third portion and a fourth portion, the fourth portion being enriched for Igs that specifically bind at least one SA capsular antigen compared to the third portion; (d) collecting the fourth portion; and (e) dissolving the Igs contained in the fourth portion in a pharmaceutically acceptable carrier. The step (b) of separating the pooled plasma into at least a first portion and a second portion can include the steps of: (b1) removing lipids from the pooled plasma to yield lipid-cleared pooled plasma; (b2) precipitating the euglobins contained in the lipid-cleared pooled plasma by dialyzing the lipid-cleared pooled plasma against a liquid selected from the group consisting of water and a low ionic strength buffer; and (b3) collecting the precipitated euglobins, wherein the second portion includes the collected euglobins. The step (b) might also further include the step (b4) of purifying IgM from the collected euglobins. In one version of this method, the step (b) of separating the pooled plasma into at least a first portion and a second portion includes the step of contacting the pooled plasma with ethanol at less than 0° C. The second portion can include Cohn fraction III paste and/or Kistler-Nitschmann precipitate B. The step (c) of separating the second portion into at least a third portion and a fourth portion includes the step of subjecting the second portion to immunoaffinity chromatography using a chromatography medium conjugated with the at least one SA capsular antigen.

Yet another method within the invention includes the steps of: (a) separating pooled plasma into a first fraction and a second fraction, wherein the first fraction includes a polyclonal mixture of Abs that is greater than 90% IgG and at least a second fraction that is at least partially depleted of IgG; and (b) isolating Igs from the at least a second fraction, wherein the isolated Igs are enriched for IgM that specifically binds at least one SA capsular antigen.

In another aspect, the invention features a method including the step of contacting a sample including Igs with (a) a chromatography medium conjugated with a SA serotype 5 capsular antigen, (b) a chromatography medium conjugated with a SA serotype 8 capsular antigen, and, optionally, (c) a chromatography medium conjugated with a non-capsular SA antigen such as Spa, the serotype 336 polysaccharide antigen, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, and Sbi.

A chromatography medium conjugated with a SA serotype 5, a SA serotype 8 capsular antigen, and, optionally, a non-capsular SA antigen such as the SA serotype 336 antigen is also featured within the invention.

The invention further includes a mAb (e.g., mammalian, human, humanized, or bovine) that specifically binds a SA capsular antigen (e.g., a serotype 5 or 8 antigen), but does not specifically bind Spa. A mixture of such mAbs is also within the invention, e.g., (i) a mixture of serotype 5 and serotype 8-specific IgM mAbs and (ii) a mixture of serotype 5,serotype 8, and serotype 336-specific IgM mAbs.

As used herein, an “antibody” or “Ab” is an Ig, a solution of identical or heterogeneous Igs, or a mixture of Igs. A “monoclonal antibody” or “mAb” is an Ab expressed by one clonal B cell line. As used herein, the term refers to a population of Ab molecules that contains only one species of an antigen binding site capable of immunoreacting with a particular epitope of a particular antigen. A “polyclonal antibody” or “polyclonal Ab” is a mixture of heterogeneous Abs. Typically, a polyclonal Ab will include myriad different Ab molecules which bind a particular antigen or particular organism with at least some of the different Abs immunoreacting with a different epitope of the antigen or organism. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the complementarity-determining regions of the heavy and light chains of the Ab). An “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

When referring to a protein molecule such as an Ab, “purified” means separated from components that naturally accompany such molecules. Typically, an Ab or protein is purified when it is at least about 10%(e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.” An Ab containing a desired Ig type and an undesired Ig type is “enriched” for the desired Ig type when treatment of the Ab results in a higher ratio of desired Ig to undesired Ig after treatment than before treatment. For example, a solution of Ab containing SA capsule-binding Igs and non-SA capsule-binding Igs is enriched for the latter when some of all of the SA capsule-binding Abs are removed from the solution; and a solution of Ab containing Spa-binding Igs and non-Spa-binding Igs is enriched for the latter when some of all of the Spa-binding Abs are removed from the solution.

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁵, 10⁶, 10⁷, 10⁸, 10⁹, 10¹¹, or 10¹² liters/mole for that other molecule.

A “therapeutically effective amount” is an amount which is capable of producing a medically desirable effect in a treated animal or human (e.g., amelioration or prevention of a disease).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the materials, methods, and examples are illustrative only and not intended to be limiting.

Other features and advantages of the invention will be apparent from the following detailed description, and from the claims.

### DETAILED DESCRIPTION

The present invention encompasses SA capsule-specific polyclonal and monoclonal IgM Ab preparations, and methods and compositions for making such Ab compositions. The below described preferred embodiments illustrate adaptations of these compositions and methods. Nonetheless, from the description of these embodiments, other aspects of the invention can be made and/or practiced based on the description provided below.

### Biological Methods

Methods involving conventional immunological and molecular biological techniques are described herein. Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Plasma fractionation methods are described in Methods of Plasma Protein Fractionation, Cursling, J. M ed., Academic Press, San Diego, Calif., 1980; and Blood Separation and Plasma Fractionation, Harris, J. R., ed., Wiley-Liss, New York 1991. Ab methods are described in Handbook of Therapeutic Abs, Dubel, S., ed., Wiley-VCH, 2007 and Harlow E. and Lane, D. Using Antibodies: A Laboratory Manual, Cold Springs Harbor Laboratory Press, Cold Spring Harbor, N.Y., 1999. Cell culture techniques are generally known in the art and are described in detail in methodology treatises such as Culture of Animal Cells: A Manual of Basic Technique, 4th edition, by R Ian Freshney, Wiley-Liss, Hoboken, N.J., 2000; and General Techniques of Cell Culture, by Maureen A Harrison and Ian F Rae, Cambridge University Press, Cambridge, UK, 1994. Methods of protein purification are discussed in Guide to Protein Purification: Methods in Enzymology, Vol. 182, Deutscher M P, ed., Academic Press, San Diego, Calif., 1990.

### Polyclonal IgM Abs Specific for SA Capsular Antigens

Conventionally, polyclonal Abs are produced by immunizing a host animal with the antigen, and later collecting the Ab-containing serum from the animal. For raising Abs specific for SA polysaccharide capsules, the antigen typically used can be one or more (1, 2, 3 or more) SA capsular polysaccharide antigens purified from bacteria. See, e.g., Ausebel et al., Current Protocols in Molecular Biology, John Wiley & Sons, Inc., NY, 1989. For inducing a T cell dependent (IgG) Ab response, the antigen(s) can be conjugated to a carrier protein such as keyhole limpet hemocyanin or pseudomonas exotoxin A. For inducing a predominantly T cell independent (IgM) Ab response, the capsular polysaccharide antigen(s) might be conjugated together to increase the average molecular weight of the antigen. An adjuvant might be used to enhance the Ab response. After one or more immunizations with the antigen, blood is collected from the host animal and the antiserum is collected by known techniques. Antigen-specific Abs contained in the antiserum can be enriched or purified by known techniques such as salt cults, ion exchange chromatography, size exclusion chromatography, and affinity chromatography.

While the foregoing method can be used in the invention, because IgM specific for SA capsular antigens occurs naturally in blood of animal subjects (e.g., humans and bovines), a presently preferred method for producing these IgMs is too simply purify them from the pooled plasma (e.g., from more than 100, 500, 1000, 5000 or more L of pooled plasma) of multiple donors (e.g., more than 5, 10, 50, 100, 500, 1000, 5000, 10000, or more individual donors) that have not been immunized as described in the preceding paragraph. This method is preferred because it does not require donor immunization and current large scale plasma fractional operations can be readily adapted to produce large quantities of IgM specific for SA capsular antigens.

Donors are preferably of the same species that the polyclonal IgM Ab composition will be administered to. For example, for administration to humans, to avoid undesired reactions, the donors are preferably human or non-human animals engineered to produce human immunoglobulins (e.g., transgenic cattle, horses, goats, or pigs expressing human heavy and light Ig chains, and having their native heavy and/or light Ig chains knocked out or down). Likewise, for administration to cows, the donors are preferably bovine. In other cases, the donors may be of a species other than human or bovine, e.g., a mammalian species such as horse, sheep, dog, cat, mouse, rat, rabbit, monkey, chimpanzee, goat, etc. To enhance recovery, donors can be specifically selected from among a general population for those that have relatively higher titers of IgM anti-SA capsule Abs [e.g., those that have 50, 100, 200, 500, 1000 or more percent greater than mean (of the general population) titer of IgM anti-SA capsule Abs; and those that have previously had a SA infection]. Donors can be tested for titers of IgM anti-SA capsule Abs using adaptations of conventional immunological techniques such as RIA and ELISA. For example, purified SA capsular antigens could be used to develop an ELISA assay.

SA capsular antigen-specific IgM can be isolated from pooled donor plasma by any suitable method. In general, such methods involve first purifying the IgMs from the plasma sample and then enriching the purified IgM product for those that specifically bind one or more SA capsular antigens. Alternatively, such methods might involve first purifying the Igs from the plasma sample that specifically bind one or more SA capsular antigens and then isolating the non-Spa binding Igs (e.g., IgMs) from the product of the first step. Further enrichment of SA capsule-specific IgMs can be performed to enrich for certain types of IgMs such as those that do not include V_{H}3 regions.

Numerous techniques for isolating IgM from plasma or another sample are known. For example, IgM may be isolated from plasma by precipitation of euglobulins (e.g., by dialysis against water or a low ionic strength buffer) followed by a size exclusion or ion exchange method. Alternatively, a modified Deutsch-Kistler-Nitschmann ethanol fractionation protocol, followed by octanoic acid precipitation and two ion-exchange chromatography steps might be used. See, Hurez et al. Blood. 1997;90:4004-13. In addition, affinity chromatography with immobilized anti-IgM Abs or immobilized C1q could be used. Other techniques for IgM purification include those described in Arnold et al., J. Biol. Chem. 280:29080-29087, 2005; U.S. Pat. Nos. 5,077,391; 5,112,952; 5,308,753; 5,612,033; and 6,136,312.

Any suitable technique for isolating Igs (e.g., IgMs) that specifically bind one or more SA capsular antigens might be used in the invention. For example, an affinity purification protocol using one or more purified SA capsular antigens as a ligand might be used. See, e.g., Antibodies: Volume 1: Production and Purification, G. Subramanian, ed., Springer 2004; Protein Purification: Principles and Practice, R. K. Scopes, Springer, 1993; and Handbook of Affinity Chromatography, T. Kline, ed., Marcel Dekker, 1993. Antigen affinity column chromatography might be used to separate Igs that specifically bind one or more SA capsular antigens from those that do not. Purified SA capasular antigens (e.g., serotype 5, 8, or a mixture of the foregoing) can be immobilized on a chromatography matrix (e.g., agarose or sepharose beads) using a joining reagent such as adipic acid dihydrazide (ADH) or N-succinimidyl-3-(2-pyridyldithio)propionate (SPDP), or the Carbolink kit (Pierce, Product #44900).

Rather than starting with donor plasma, in an alternative embodiment, commercially available Ig preparations (IVIGs) that contain IgM might be screened for IgMs that specifically bind one or more SA capsular antigens. Lots containing such antibodies can be used as a source from which such SA-capsule specific IgMs can be purified. A number of IVIG products are presently available in the commercial marketplace including, e.g., Flebogamma® (Grifols), Gamunex™ (Talecris Biotherapeutics), Octagam® (Octapharma), Gammagard® (Baxter Healthcare), Carimune™ NF (CSL Behring), Gammar® P (CSL Behring), Iveegam EN (Baxter Healthcare), Panglobulin® NF (Baxter Healthcare), Polygam® S/D (Baxter Healthcare), Sandoglobulin ® (CSL Behring), and Pentaglobin® (Biotest AG). Those that have higher concentrations of IgM are preferred (e.g., Pentaglobin® WIG). Because most commercial WIG production operations are primarily directed to purifying the IgG fraction of plasma, IgG-depleted waste products of such operations that contain IgM might be used as starting materials for the purification methods described herein.

The purified Ab compositions of the invention might include plasma components other than SA capsular antigen-specific IgM Abs. For example, the compositions might include IgGs, IgAs, IgEs, albumin, etc. Preferably, to ensure that high titers of SA capsular antigen-specific IgM can be administered to a subject with minimal adverse effects, the Ab compositions of the invention preferably include at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight SA capsular antigen-specific IgMs. In addition to SA capsular antigen-specific IgMs, the antibody compositions of the invention might also include Igs (which may be Spa-binding or non-Spa binding Igs such as IgMs) that specifically bind one or more SA antigens other than a capsular antigen. Examples of such other antigens include Spa, serotype 336 polysaccharide antigen, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, and Sbi.

### Monoclonal IgM Abs Specific for SA Capsular Antigens

In addition to polyclonal Abs, the invention also features monoclonal IgM Abs that specifically bind one or more SA capsular antigens. Because B lymphocytes which express IgM specific for SA capsular antigens occur naturally in humans and other animals such as cows, a presently preferred method for raising mAbs is to first isolate such a B lymphocyte from a subject and then immortalize it so that is can be continuously replicated in culture. Subjects lacking large numbers of naturally occurring B lymphocytes which express IgM specific for SA capsular antigens may be immunized with one or more polysaccharide SA capsule antigens or may be infected with a sublethal amount of SA the express one or more polysaccharide SA capsule antigens to increase the number of such B lymphocytes. Human mAbs are prepared by immortalizing a human antibody secreting cell (e.g., a human plasma cell). See, e.g., U.S. Pat. No. 4,634,664. Bovine mAbs can be prepared by adapting known methods such as those described in U.S. Pat. No. 5,087,693.

In an exemplary method, one or more (e.g., 5, 10, 25, 50, 100, 1000, or more) human subjects (e.g., subjects not previously administered an SA vaccine) are screened for the presence of such IgM in their blood. Those subjects that express the desired IgM Ab can then be used as B lymphocyte donors. In one possible method, peripheral blood is obtained from a human donor that possesses B lymphocytes that express IgM specific for one or more SA capsular antigens. Such B lymphocytes are then isolated from the blood sample, e.g., by cells sorting (e.g., fluorescence activated cell sorting, “FACS”; or magnetic bead cell sorting) to select IgM⁺ B lymphocytes (or IgM⁺ CD27⁺ memory B lymphocytes). These cells can then be immortalized by viral transformation (e.g., using EBV) or by fusion to another immortalized cell such as a human myeloma according to known techniques. The B lymphocytes within this population that expresses IgM specific for an SA capsular antigen can then be isolated by limiting dilution methods (e.g., cells in wells of a microtiter plate that are positive for IgM specific for an SA capsular antigen are selected and subcultured, and the process repeated until a desired clonal line can be isolated). See, e.g., Goding, Monoclonal Antibodies: Principles and Practice, pp. 59-103, Academic Press, 1986. Those clonal cell lines that express IgM having at least nanomolar or picomolar binding affinities for an SA capsular antigen are preferred. MAbs secreted by these clonal cell lines can be purified from the culture medium, ascites fluid, or serum by conventional immunoglobulin purification procedures such as salt cuts, size exclusion, ion exchange separation, and affinity chromatography.

Because B lymphocytes that express IgM specific for one or more SA capsular antigens are believed to be present in relatively high amounts in certain subjects, SA capsular antigen conjugated with a detectable label might be used to identify and select those particular B lymphocytes from among the general B lymphocyte population. For example, B lymphocytes that express a SA capsular antigen-binding IgM might be isolated from a peripheral blood sample using FACS with an anti-IgM Ab labeled with a first fluorophore and a SA-capsular antigen labeled with a second fluorophore, and optionally an anti-CD27⁺ Ab labeled with a third fluorophore. The B lymphocytes isolated according to this method can then be immortalized and further selected as described above.

Although immortalized B lymphocytes might be used in in vitro cultures to directly produce mAbs, in certain cases it might be desirable to use heterologous expression systems to produce mAbs. See, e.g., the methods described in U.S. patent application Ser. No. 11/754,899. For example, the genes encoding an IgM mAb specific for one or more SA capsular antigens might be cloned and introduced into an expression vector (e.g., a plasmid-based expression vector) for expression in a heterologous host cell (e.g., CHO cells, COS cells, myeloma cells, and E. coli cells). Because Igs include heavy (H) and light (L) chains in an H₂L₂ configuration, the genes encoding each may be separately isolated and expressed in different vectors. In addition, a gene encoding the IgM J (“joining”) chain might be co-expressed along with the heavy and light chain genes to mimic the situation in native B lymphocytes and produce mostly pentameric IgM. On the other hand, in some cases it might be preferred not to co-express a J chain, e.g., where it is desirable to produce mostly hexameric IgM (which can be more effective at activating complement). See, e.g., Randall et al., J. Biol. Chem. 267:18002-18007, 1992; Davis et al., EMBO J. 8:2519-2526, 1998.

Although generally less preferred, chimeric mAbs (e.g., “humanized” mAbs), which are antigen-binding molecules having different portions derived from different animal species (e.g., variable region of a mouse immunoglobulin fused to the constant region of a human immunoglobulin), might be used in the invention. Such chimeric antibodies can be prepared by methods known in the art. E.g., Morrison et al., Proc. Nat'l. Acad. Sci. USA, 81:6851, 1984; Neuberger et al., Nature, 312:604, 1984; Takeda et al., Nature, 314:452, 1984. Similarly, antibodies can be humanized by methods known in the art. For example, monoclonal antibodies with a desired binding specificity can be commercially humanized or as described in U.S. Pat. Nos. 5,693,762; 5,530,101; or 5,585,089.

The mAbs described herein might be affinity matured to enhance or otherwise alter their binding specificity by known methods such as VH and VL domain shuffling (Marks et al. Bio/Technology 10:779-783, 1992), random mutagenesis of the hypervariable regions (HVRs) and/or framework residues (Barbas et al. Proc Nat. Acad. Sci. USA 91:3809-3813, 1994; Schier et al. Gene 169:147-155, 1995; Yelton et al. J. Immunol. 155:1994-2004, 1995; Jackson et al., J. Immunol. 154(7):3310-9, 1995; and Hawkins et al, J. Mol. Biol. 226:889-896, 1992. Amino acid sequence variants of an Ab may be prepared by introducing appropriate changes into the nucleotide sequence encoding the Ab. In addition, modifications to nucleic acid sequences encoding mAbs might be altered (e.g., without changing the amino acid sequence of the mAb) for enhancing production of the mAb in certain expression systems (e.g., intron elimination and/or codon optimization for a given expression system). The mAbs described herein can also be modified by conjugation to another protein (e.g., another mAb) or non-protein molecule. For example, a mAb might be conjugated to a water soluble polymer such as polyethylene glycol or a carbon nanotube (See, e.g., Kam et al., Proc. Natl. Acad. Sci. USA 102: 11600-11605, 2005). See, U.S. patent application Ser. No. 11/754,899.

Preferably, to ensure that high titers of SA capsular antigen-specific IgM can be administered to a subject with minimal adverse effects, the mAb compositions of the invention are at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight pure (excluding any excipients). The mAb compositions of the invention might include only a single type of mAb (i.e., one produced from a single clonal B lymphocyte line) or might include a mixture of two or more (e.g., 2, 3, 4, 5, 6, 7, 8, 9, 10 or more) different types of mAbs (e.g., a composition including a first mAb specific for a type 5 capsular antigen, a second mAb specific for a type 8 capsular antigen, and/or a third mAb specific for a type 336 antigen; or a composition including first mAb specific for a first epitope of a type 5 capsular antigen, a second mAb specific for a second epitope of the type 5 capsular antigen, and/or a third mAb specific for a third epitope of the type 5 capsular antigen, wherein the first, second, and third epitope each differ from one another). IgMs that utilize a V_{H} other than V_{H}3 are preferred in applications where V_{H}3-protein A interaction is undesirable. In addition to SA capsular antigen-specific IgM mAbs, the antibody compositions of the invention might also include other mAbs (which may be Spa-binding or, preferably non-Spa binding Igs such as IgMs) that specifically bind one or more SA antigens other than a capsular antigen. Examples of such other antigens include Spa, a type 336 antigen, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, and Sbi.

### Other Bacterial Targets

The compositions of the invention might also include antibodies (e.g., IgMs or other Igs lacking a Fc region that binds SpA) that specifically bind one or more bacteria other than SA. Such compositions might be particularly useful to treat subjects suspected of simultaneously being infected with SA and one or more non-SA bacteria. Thus various compositions of the invention can include antibodies that can specifically bind one or more antigens from the following species or strains: might Acinetobacter baumanii (Family Moraxellaceae); Actinobacillus spp. (Family Pasteurellaceae), Actinomycetes(actinomycetes, streptomycetes); Actinomyces such as Actinomyces israelii, Actinomyces naeslundii, and Actinomyces spp.; Aeromonas spp. (Family Aeromonadaceae) such as Aeromonas hydrophila, Aeromonas veronii biovar sobria (Aeromonas sobria), and Aeromonas caviae; Peptostreptococcus spp.; Streptococcus spp.; Veillonella spp.; Mobiluncus spp.; Propionibacterium acnes; Lactobacillus spp.; Eubacterium spp.; Bifidobacterium spp.; Bacteroides spp.; Prevotella spp.; Porphyromonas spp.; Fusobacterium spp.; Bacillus spp. (Family Bacillaceae) such as Bacillus anthracis, Bacillus cereus, Bacillus subtilis, Bacillus thuringiensis, and Bacillus stearothermophilus; Bacteroides spp. (Family Bacteroidaceae) such as Bacteroides fragilis; Bordetella spp. Including Bordetella pertussis, Bordetella parapertussis, and Bordetella bronchiseptica; Borrelia spp. (Order Spirochaetales; Family Spirochaetaceae) such as Borrelia recurrentis and Borrelia burgdorferi; Brucella spp. including Brucella abortus, Brucella canis, Brucella melintensis, and Brucella suis; Burkholderia spp. such as Burkholderia pseudomallei and Burkholderia cepacia; Campylobacter spp. Including Campylobacter jejuni, Campylobacter coli, Campylobacter lari, and Campylobacter fetus; Citrobacter spp. (Family Enterobacteriaceae); Clostridium spp. Such as Clostridium perfringens, Clostridium difficile, and Clostridium botulinum; Chlamydia spp. such as Chlamydia tachomatis and Chlamydia pneumonia; Corynebacterium spp. including Corynebacterium diphtheria, Corynebacterium jeikeum, and Corynebacterium urealyticum; Edwardsiella tarda; Enterobacter spp. including Citrobacter freundii, Citrobacter diversus, Enterobacter aerogenes, Enterobacter agglomerans, and Enterobacter cloacae; Escherichia coli; Klebsiella spp. such as Klebsiella pneumoniae and Klebsiella oxytoca; Morganella morganii; Mycoplasma pneumonia; Proteus spp. including Proteus mirabilis and Proteus vulgaris; Providencia spp. such as Providencia alcalifaciens, Providencia rettgeri, and Providencia stuartii; Rickettsia spp. such as Rickettsia rickettsii; Salmonella spp. including Salmonella enterica, Salmonella typhi, Salmonella paratyphi, Salmonella enteritidis, Salmonella cholerasuis, and Salmonella typhimurium; Serratia spp. such as Serratia marcesans and Serratia liquifaciens; Shigella spp. including Shigella dysenteriae, Shigella flexneri, Shigella boydii, and Shigella sonnei; Yersinia spp. such as Yersinia enterocolitica, Yersinia pestis, and Yersinia pseudotuberculosis; Enterococcus spp. such as Enterococcus faecalis and Enterococcus faecium; Erysipelothrix rhusopathiae; Francisella tularensis; Haemophilus spp. (Family Pasteurellaceae) such as Haemophilus influenza, Haemophilus ducreyi, Haemophilus aegyptius, Haemophilus parainfluenzae, Haemophilus haemolyticus, and Haemophilus parahaemolyticus; Helicobacter spp. including Helicobacter pylori, Helicobacter cinaedi, and Helicobacter fennelliae; Legionella pneumophila; Leptospira interrogans; Listeria monocytogenes; Micrococcus spp. (Family Micrococcaceae); Moraxella catarrhalis; Mycobacterium spp. such as Mycobacterium leprae and Mycobacterium tuberculosis; Nocardia spp. including Nocardia asteroides and Nocardia brasiliensis; Neisseria spp. (Family Neisseriaceae) such as Neisseria gonorrhoeae and Neisseria meningitides; Pasteurella multocida; Plesiomonas shigelloides; Propionibacterium acnes; Pseudomonas aeruginosa; Rhodococcus spp.; Staphylococcus spp. including Staphylococcus aureus, Staphylococcus epidermidis, and Staphylococcus saprophyticus; Stenotrophomonas maltophilia; Streptococcus pneumonia; Streptococcus spp. such as Streptococcus pyogenes, Streptococcus agalactiae, Streptococcus anginosus, Streptococcus equismilis, Streptococcus bovis, and Streptococcus mutans; Streptomyces spp.; Treponema spp. including Treponema pallidum ssp. pallidum, Treponema pallidum ssp. Endemicum, Treponema pallidum ssp. Pertenue, and Treponema carateum; and Vibrio spp. (Family Vibrionaceae) such as Vibrio cholera, Vibrio parahaemolyticus, Vibrio vulnificus, Vibrio alginolyticus, Vibrio mimicus, Vibrio hollisae, Vibrio fluvialis, Vibrio metchnikovii, Vibrio damsel, and Vibrio furnish.

Examples of bacterial antigens which the antibodies of the invention can specifically bind include capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype O11); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1 6 glucosamine, catalase, beta-lactamase, teichoic acid, peptidoglycan, a penicillin binding protein, chemotaxis inhibiting protein, complement inhibitor, Sbi, and von Willebrand factor binding protein.

### Pharmaceutical and Diagnostic Compositions and Methods

The Ab compositions of the invention may be administered to animals or humans in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Abs compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and or 0.2 micron filter); contacted with beta-propiolactone; and/or dissolved in a solution including a microbicide (e.g., a detergent, an organic solvent, and a mixture of a detergent and organic solvent). In addition the SA-capsule specific IgMs of the invention might be monomeric, dimeric, pentameric, heptameric, conjugated with an antibiotic (see U.S. Pat. No. 5,545,721), polyethylene glycol, or detectable label such as biotin, fluorophore or radioisotope.

The compositions of the invention may be administered to animals or humans by any suitable technique. Typically, such administration will be parenteral (e.g., intravenous, subcutaneous, intramuscular, or intraperitoneal introduction). The compositions may also be administered directly to the target site (e.g., an abscess) by, for example, surgical delivery to an internal or external target site, or by catheter to a site accessible by a blood vessel. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. It is expected that an appropriate dosage for intravenous administration of antibodies would be in the range of about 0.01 to 100 mg/kg body weight.

The Ab compositions of the invention might be used to prevent a subject from being developing a SA infection (e.g., an antibiotic-resistant SA infection such as one caused by a methicillin- or vancomycin-resistant strain). Examples of subjects at high risk for developing a SA infection include those who are admitted to a hospital, those that will be or have been subjected to an invasive medical procedure such as surgery or catheterization, and those who are immunosuppressed. Although Abs specific for only a singular type of SA capsule (e.g., type 5 or 8) might be used to prevent an SA infection, because it is difficult to project which exact SA serotype might infect a subject, for prophylaxis it is preferred to use an Ab composition containing Abs specific for the most prevalent serotypes of SA that infect a given species. For example, for human subjects, a prophylactic Ab composition might include Abs specific for at least the 5 and 8 serotype (e.g., one containing Abs specific for serotype 5, 8, and 336 and any other serotype that is clinically relevant). The Ab compositions of the invention might also be used to treat a subject with an existing SA infection (e.g., an antibiotic-resistant SA infection such as one caused by a methicillin- or vancomycin-resistant strain). While Abs specific for multiple types of SA capsule might be used to treat an SA infection, it is generally preferred to first serotype the particular SA causing the infection and then administer to the subject only those Abs that bind only that serotype. For instance, if a patient is infected with only type 5 SA, then only Abs specific for type 5 need to be administered.

The SA-specific Abs described herein might also be used in devices and methods for detecting a SA infection wherein a sample suspected of harboring SA is contacted with an Ab or Abs specific for one or more SA capsular antigens, and any SA-Ab reactions are detected to diagnose the presence of SA. The lack of Spa binding by IgM Abs allows discrimination between specific antigen binding and non-specific Spa binding. Thus, the Abs can be used to specifically detect the serotype of SA present in a sample. In certain embodiments, the Abs are conjugated with a detectable label such as an enzyme, a metal particle, a fluorophore, a dye, a nanoparticle, and/or a radioisotope. In some cases, a secondary antibody specific for the anti-SA antibody might be used to detect the anti-SA antibody, e.g., in an ELISA, RIA, or immunoprecipitation assay.

### Other Embodiments

It is to be understood that while the invention has been described in conjunction with the detailed description thereof, the foregoing description is intended to illustrate and not limit the scope of the invention, which is defined by the scope of the appended claims. For example, non-staphylococcal protein A (Spa)-binding Igs other than IgM might be used (e.g., a human IgG₃ with arginine at amino acid position 435); SA capsular antigen-based vaccines may be formulated to preferentially provoke a predominantly IgM (as opposed to IgG) response; Abs of the invention maybe specific for capsular antigens of encapsulated bacterial species that express an Fc-binding protein (e.g., protein G) other than SA; and the compositions described herein might also be used in conjunction with other agents such as conventional antibiotics and/or inhibitors of other SA virulence factors (e.g., inhibitors of catalase or other SA antioxidants). Other aspects, advantages, and modifications are within the scope of the following claims.

I claim:

1. A composition comprising purified antibodies and a pharmaceutically acceptable carrier, wherein IgMs that specifically bind at least one Staphylococcus aureus capsular antigen comprise at least about 5 percent by weight of the antibodies in the composition.

2. The composition of claim 1, wherein the IgMs comprises at least about 50 percent by weight of the antibodies in the composition.

3. The composition of claim 1, wherein the composition comprises IgMs that specifically bind Staphylococcus aureus serotype 5 capsular antigen and IgMs that specifically bind Staphylococcus aureus serotype 8 capsular antigen.

4. The composition of claim 1, wherein the antibodies also comprise immunoglobulins that specifically bind a staphylococcal antigen other than a capsular antigen.

5. The composition of claim 4, wherein the staphylococcal antigen other than a capsular antigen is one selected from the group consisting of: protein A, a serotype 336 polysaccharide antigen, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, and Sbi.

6. The composition of claim 1, wherein the IgMs are fully human.

7. The composition of claim 1, wherein the IgMs comprise both human and non-human immunoglobulin portions.

8. The composition of claim 1, wherein the IgMs are purified from pooled plasma.

9. The composition of claim 8, wherein the plasma is human.

10. The composition of claim 8, wherein the plasma is bovine.

11. A composition comprising purified antibodies and a pharmaceutically acceptable carrier, the purified antibodies comprising at least about 5 percent by weight a human or humanized IgM monoclonal antibody that specifically binds at least one Staphylococcus aureus capsular antigen but does not specifically bind staphylococcal protein A.

12. The composition of claim 11, wherein the IgM monoclonal antibody comprises at least about 50 percent by weight of the antibodies in the composition.

13. The composition of claim 11, wherein the antibodies comprise IgMs that specifically bind Staphylococcus aureus serotype 5 capsular antigen and IgMs that specifically bind Staphylococcus aureus serotype 8 capsular antigen.

14. The composition of claim 11, wherein the antibodies also comprise immunoglobulins that specifically bind a staphylococcal antigen other than a capsular antigen.

15. The composition of claim 14, wherein the staphylococcal antigen other than a capsular antigen is one selected from the group consisting of: protein A, a serotype 336 polysaccharide antigen, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, and Sbi.

16. The composition of claim 11, wherein the IgMs are fully human.

17. The composition of claim 11, wherein the IgMs comprise both human and non-human immunoglobulin portions.

18. A method comprising the steps of:
isolating from pooled plasma a polyclonal mixture of antibodies that is enriched for IgMs that specifically bind at least one Staphylococcus aureus capsular antigen; and
dissolving the isolated polyclonal mixture of antibodies in a pharmaceutically acceptable carrier.

19. A method comprising the steps of:
(a) obtaining at least 10 liters of plasma pooled from at least ten donors;
(b) separating the pooled plasma into at least a first portion and a second portion, the second portion being enriched for IgMs compared to the first portion;
(c) separating the second portion into at least a third portion and a fourth portion, the fourth portion being enriched for IgMs that specifically bind at least one Staphylococcus aureus capsular antigen compared to the third portion;
(d) collecting the fourth portion; and
(e) dissolving the IgMs contained in the fourth portion in a pharmaceutically acceptable carrier.
